(19) United States
(12) Patent Application Publication
(10) Pub. No.: US 2026/0294518 A1
(43) Pub. Date: Oct. 1, 2026

(54) ELECTROSURGICAL FORCEPS INCLUDING A THICK TISSUE SENSOR

(71) Applicant: Covidien LP, Mansfield, MA (US)

(72) Inventors: Robert F. MCCULLOUGH, Jr., Boulder, CO (US); Hayden M. MAY, Westminster, CO (US); Jennifer L. RICH, Parker, CO (US); Kelley D. GOODMAN, Erie, CO (US); David D. BRAUSE, Longmont, CO (US); Caitlin K. YAMAKA, Westminster, CO (US); Jenna DANCY, Longmont, CO (US); Gregory W. FISCHVOGT, Reno, NV (US); Mark A. JOHNSTON, Boulder, CO (US); Lewis R. PUTERBAUGH, Longmont, CO (US)

(73) Assignee: Covidien LP, Mansfield, MA (US)

(21) Appl. No.: 19/489,141

(22) PCT Filed: May 31, 2024
(86) PCT No.: PCT/IB2024/055334

§ 371 Date: Dec. 1, 2025

Related U.S. Application Data
(60) Provisional application No. 63/470,475, filed on Jun. 2, 2023.

(51) Int. Cl. A61B 18/14; A61B 18/00; A61B 18/12

(52) U.S. Cl. CPC A61B 18/1445; A61B 18/1206; A61B 2018/00178; A61B 2018/00184; A61B 2018/0063; A61B 2018/00702; A61B 2018/00779; A61B 2018/00875

(57) ABSTRACT

A surgical instrument includes a housing, a shaft, an end effector assembly, a drive assembly, and a tissue sensor assembly. The end effector assembly has a first jaw and a second jaw movable relative to one another between an open configuration wherein the first and second jaws define a first jaw aperture angle greater than a pre-determined jaw aperture angle and a closed configuration wherein the first and second jaws define a second jaw aperture angle less than the pre-determined jaw aperture angle. The drive assembly includes a drive tube extending through the shaft that is configured to move the first and second jaws between the open and closed configurations. The tissue sensor assembly includes a tissue flag coupled to the drive assembly and translatable with the drive tube, and a tissue flag sensor coupled to the housing for detecting movement of the tissue flag.

### CROSS REFERENCE TO RELATED APPLICATIONS

This application claims the benefit of U.S. Provisional Application No. 63/470,475, filed on Jun. 2, 2023, which application is incorporated herein by reference in its entirety.

### FIELD

This disclosure generally relates to electrosurgical forceps. More particularly, this disclosure relates to a thick tissue sensor for use with an electrosurgical forceps for sealing, cutting, and/or coagulating tissue.

### BACKGROUND

Electrosurgical forceps utilize both mechanical clamping action and electrical energy to affect hemostasis by heating tissue and blood vessels to coagulate, cauterize and/or seal the tissue.

Many surgical procedures require cutting or ligating blood vessels or vascular tissue. By utilizing an electrosurgical forceps, a surgeon can cauterize, coagulate/desiccate, and/or simply reduce or slow bleeding simply by controlling the intensity, frequency and duration of the electrosurgical energy applied through the jaws to the tissue.

Effectively sealing vessels or tissue may be achieved by accurately controlling the pressure applied to the vessel or tissue and the gap distance between the electrodes-both of which are affected by the thickness of the sealed vessel or tissue. The pressure applied to the vessel or tissue may be controlled by the mechanical design of the instrument.

It can be difficult for surgeons to visually determine the gap distance between electrodes before energy application. After energy application, it may be difficult to ensure that the jaws of the forceps have achieved an appropriate seal closure. The visualization of the surgical field may be difficult because of blood within the surgical field, lack of complete vessel or tissue dissection, or isolation.

### SUMMARY

Surgical instruments described herein deliver improved performance over conventional surgical instruments on thick or challenging tissue and/or large vessels. The surgical instruments of this disclosure include an identifier circuit that identifies when proper sealing pressure is achieved and enables communication to a user if a seal attempt was successful or unsuccessful (e.g., when to transect). In aspects, the identifier circuit checks the status of the jaws of the apparatus to ensure adequate tissue compression and, in some aspects, the apparatus includes a tissue sensor assembly that detects the jaw aperture angle to determine a thickness of tissue disposed between the jaws.

In accordance with aspects of this disclosure, a surgical instrument includes a housing, a shaft extending distally from the housing, and an end effector assembly disposed at a distal end of the shaft. The end effector assembly includes a first jaw and a second jaw movable relative to one another between an open configuration wherein the first and second jaws define a first jaw aperture angle greater than a pre-determined jaw aperture angle and a closed configuration wherein the first and second jaws define a second jaw aperture angle less than the pre-determined jaw aperture angle. The surgical instrument further includes a drive assembly and a tissue sensor assembly. The drive assembly includes a drive tube extending through the shaft and configured to move the first and second jaws between the open and closed configurations. The tissue sensor assembly includes a tissue flag coupled to the drive assembly and translatable with the drive tube, and a tissue flag sensor coupled to the housing for detecting movement of the tissue flag.

The tissue sensor assembly may be configured to change states when the first and second jaws are moved between the open and closed configurations. In some aspects, the tissue sensor assembly is in an open state when the first and second jaws are in the open configuration, and the tissue sensor assembly is in a closed state when the first and second jaws are in the closed configuration. In aspects, when the first and second jaws are in the open configuration, the tissue flag is disposed in spaced relation from the tissue flag sensor and, when the first and second jaws are in the closed configuration, the tissue flag contacts the tissue flag sensor.

The surgical instrument may further include an identifier circuit configured to identify when the first and second jaws are in the open configuration or the closed configuration. The surgical instrument may further include an activation assembly configured to deploy energy to the first and second jaws. In some aspects, the identifier circuit may be configured to identify when energy is deployed to the first and second jaws. An output of the identifier circuit may be used to determine the quality of a seal generated by the activation assembly.

In aspects, the identifier circuit enables four states of the surgical instrument to be monitored based on an open state or a closed state of each of the tissue sensor assembly and the activation assembly. In some aspects, the identifier circuit includes a plurality of resistors, a first switch associated with the tissue sensor assembly, and a second switch associated with the activation assembly, wherein the first and second switches determine a connection scheme of the plurality of resistors. The identifier circuit may generate an identifier circuit impedance based on each of the four states of the surgical instrument. In some aspects, the identifier circuit impedance is read through an output terminal of the identifier circuit. The output terminal may be configured to communicate with a generator. In certain aspects, the surgical instrument further includes an electrosurgical cable extending from the housing. The electrosurgical cable includes a plug connectable to a generator and the output terminal is a pin of the plug.

In accordance with aspects of this disclosure, a surgical instrument includes a housing, a shaft extending distally from the housing, and an end effector assembly disposed at a distal end of the shaft. The end effector assembly includes a first jaw and a second jaw movable relative to one another between an open configuration wherein the first and second jaws define a first jaw aperture angle greater than a pre-determined jaw aperture angle and a closed configuration wherein the first and second jaws define a second jaw aperture angle less than the pre-determined jaw aperture angle. The surgical instrument further includes a tissue sensor assembly, an activation assembly, and an identifier circuit. The tissue sensor assembly has an open state when the first and second jaws are in the open configuration and a closed state when the first and second jaws are in the closed configuration. The activation assembly has an open state when no energy is deployed to the first and second jaws and a closed state when energy is deployed to the first and second jaws. The identifier circuit is configured to identify four states of the surgical instrument based on the open state or the closed state of each of the tissue sensor assembly and the activation assembly.

In aspects, the identifier circuit includes a plurality of resistors, a first switch associated with the tissue sensor assembly, and a second switch associated with the activation assembly, wherein the first and second switches determines a connection scheme of the plurality of resistors. The identifier circuit may output an identifier circuit impedance based on each of the four states of the surgical instrument. In some aspects, the identifier circuit impedance is used to determine the quality of a seal generated by the activation assembly.

In aspects, the surgical instrument further includes a drive assembly configured to move the first and second jaws between the open and closed configurations, and the tissue sensor assembly includes a tissue flag movably coupled to the drive assembly and a tissue flag sensor for detecting movement of the tissue flag. In some aspects, the tissue flag is aligned with the tissue flag sensor such that when the first and second jaws are in the open configuration, the tissue flag is disposed in spaced relation from the tissue flag sensor and, which the first and second jaws are in the closed configuration, the tissue flag contacts the tissue flag sensor.

The details of one or more aspects of this disclosure are set forth in the accompanying drawings and the description below. Other aspects, as well as features, objects, and advantages of the aspects described in this disclosure will be apparent from the description and drawings, and from the claims.

### BRIEF DESCRIPTION OF THE DRAWINGS

Various aspects of this disclosure are described hereinbelow with reference to the drawings, which are incorporated in and constitute a part of this specification, wherein:

FIG. 1 is a side view of an electrosurgical forceps in accordance with aspects of this disclosure;

FIG. 2 is an internal, side, cutaway view of the electrosurgical forceps of FIG. 1, showing a handle assembly and a drive assembly in an initial position;

FIG. 3 is a close-up view of the area of detail indicated in FIG. 2, showing a tissue sensor assembly of the electrosurgical forceps;

FIG. 4 is a cross-sectional view of the electrosurgical forceps of FIG. 2, taken along section line 4-4 of FIG. 2, showing the tissue sensor assembly in an open state;

FIG. 5 is a perspective view of a tissue flag of the tissue sensor assembly of the electrosurgical forceps of FIG. 4;

FIG. 6 is a perspective view of a tissue flag sensor of the tissue sensor assembly of the electrosurgical forceps of FIG. 4;

FIG. 7 is a perspective view of an end effector assembly of the electrosurgical forceps of FIG. 1, showing first and second jaws of the end effector assembly in an open configuration;

FIG. 8 is a close-up view of the tissue sensor assembly of the electrosurgical forceps of FIG. 4, showing the tissue flag contacting the tissue flag sensor during actuation of the drive assembly of the electrosurgical forceps;

FIG. 9 is a close-up view of the tissue sensor assembly of the electrosurgical forceps of FIG. 4, showing the tissue sensor assembly in a closed state;

FIG. 10 is a side view of the end effector assembly of the electrosurgical forceps of FIG. 1, showing the first and second jaws in a partially closed configuration corresponding to the closed state of the tissue sensor assembly of FIG. 9;

FIG. 11 is a side view of the end effector assembly of the electrosurgical forceps of FIG. 10, showing the first and second jaws in a further closed configuration;

FIG. 12 is a schematic diagram of the electrosurgical forceps of FIG. 1, showing a resistor or identifier circuit of the electrosurgical forceps in accordance with aspects of this disclosure;

FIG. 13 is a schematic diagram of the electrosurgical forceps of FIG. 1, showing a resistor or identifier circuit of the electrosurgical forceps in accordance with other aspects of this disclosure; and

FIG. 14 is a schematic view of a robotic surgical system in accordance with aspects of this disclosure.

### DETAILED DESCRIPTION

Aspects of this disclosure will now be described in detail with reference to the drawing figures wherein like reference numerals identify similar or identical elements. Throughout this description, the term “proximal” refers to a portion or component of a structure that is closer to a user, and the term “distal” refers to a portion or component of the structure that is farther from the user.

It should be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

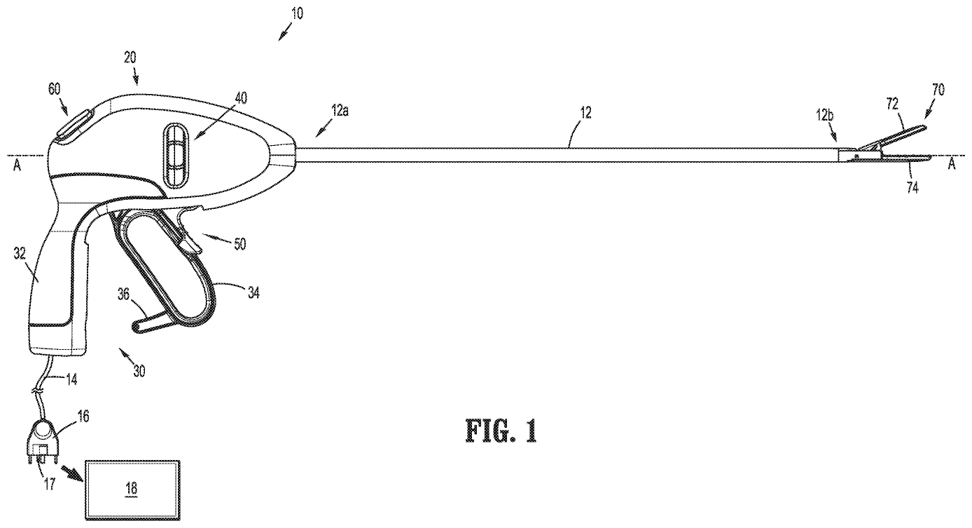

Referring now to FIG. 1, an electrosurgical forceps 10 (also referred to herein generally as a forceps) is provided in accordance with aspects of this disclosure and includes a housing 20, a handle assembly 30, a rotating assembly 40, a trigger assembly 50, an activation assembly 60, and an end effector assembly 70. The end effector assembly 70 includes first and second jaws 72, 74 for grasping, sealing, and treating vessels and vascular tissue. For the purposes herein, the forceps 10 will be described generally. However, for a detailed description of the structure and function of exemplary forceps suitable for use with aspects of this disclosure, reference may be made to U.S. Patent Appl. Pub. No. 2022/0183746, the entire contents of which are incorporated by reference herein. It should be appreciated that principles of this disclosure are equally applicable to forceps having other configurations.

The forceps 10 includes a shaft 12 that defines a longitudinal axis “A-A” of the forceps 10. A proximal end portion 12a of the shaft 12 is operatively engaged to the housing 20 and a distal end portion 12b of the shaft 12 is configured to mechanically engage the end effector assembly 70 to move the first and second jaws 72, 74 between an open configuration (FIG. 1), a closed configuration (FIG. 11), and a plurality of partially closed configurations between the open configuration and the closed configurations.

The forceps 10 includes an electrosurgical cable 14 having a plug or connector 16 that connects the forceps 10 to a source of electrosurgical energy, e.g., a generator 18. The connector 16 is configured to mate with a port (not shown) on the generator 18. The generator 18 provides electrosurgical energy to the end effector assembly 70 of the forceps 10. It is also contemplated that the forceps 10 may include an energy source, e.g., a battery and generator (not shown) positioned on or within the housing 20 to provide electrosurgical energy to the forceps 10.

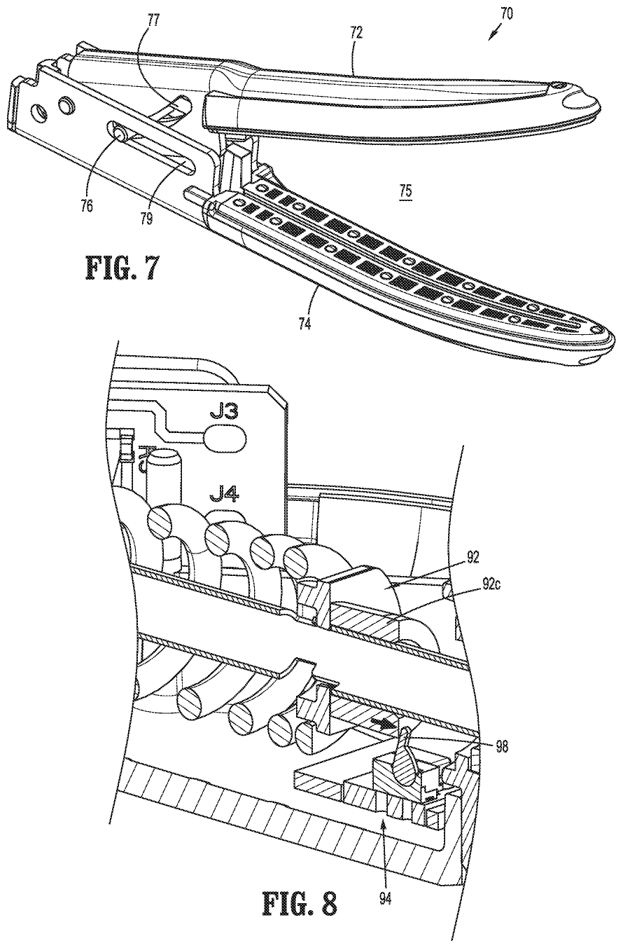

Referring to FIGS. 1 and 2, the handle assembly 30 includes a fixed handle 32 and a moveable handle 34. The fixed handle 32 is integrally associated with housing 20 and the moveable handle 34 is movable relative to the fixed handle 32 to actuate a drive assembly 80 (FIG. 2). The moveable handle 34 has an upper end portion 34a that is pivotally secured within the housing 20 and operably engaged with the drive assembly 80 (FIG. 2) of the forceps 10. Briefly describing operation of the drive assembly 80, the moveable handle 34 operably couples to a series of links 82 that cooperate to move a drive mechanism 84 (e.g., a carriage) distally against a compression spring 86 which, in turn, regulates movement of a drive tube 88 that translates through the shaft 12 and regulates the overall closure force of the first jaw 72 relative to the second jaw 74. More particularly, as seen in FIGS. 2 and 7, the translation of the drive tube 88 controls the movement of a cam pin 76 within cam slots 77, 79 defined respectively within the first and second jaws 72 and 74, which, in turn, forces the first jaw 72 to move or pivot relative to the second jaw 74 when pushed distally to grasp tissue disposed within a space 75 defined between the first and second jaws 72, 74. It should be understood that while the drive assembly 80 is described above as having a push-to-close configuration to actuate the first and second jaws 72, 74, other configurations are envisioned, such as a pull-to-close configuration.

With continued reference to FIGS. 1 and 2, the movable handle 34 includes a flange 36 extending proximally from a lower end portion 34b of the movable handle 34. The flange 36 is configured to engage an aperture 31 defined within the fixed handle 32 and ultimately engage a latch 38 that is configured to selectively lock and unlock the fixed and movable handles 32, 34 relative to one another as needed during surgery. Upon initial movement and engagement of the flange 36 into the aperture 31 with the latch 38, the fixed and movable handles 32, 34 will lock with one another to hold the first and second jaws 72, 74 in a closed position (e.g., during vessel sealing and cutting), and upon subsequent movement relative to one another, the fixed and movable handles 32, 34 would unlock.

The rotating assembly 40 is disposed substantially within the housing 20 and enables a user to control the orientation of the shaft 12 and thus, the first and second jaws 72, 74. In aspects, the rotating assembly 40 is infinitely rotatable in either direction about the longitudinal axis “A-A” to similarly rotate the end effector assembly 70 relative to the housing 20. Alternatively, the rotating assembly 40 may have a defined range of motion. The trigger assembly 50 enables a user to deploy a blade (not shown) between the first and second jaws 72, 74 to divide tissue independent of tissue sealing.

The activation assembly 60 enables a user to deploy energy to the first and second jaws 72, 74 for sealing tissue. The activation assembly 60 includes an activation button 62 incorporated into a body 22 of the housing 20 that is operably coupled to a main printed circuit board assembly 64 to enable energy to be activated by a user and delivered to the first and second jaws 72, 74 as controlled by a generator algorithm. In aspects, the generator algorithm checks and adjusts energy delivery to tissue based upon reading tissue impedance between the first and second jaws 72, 74. In some aspects, the activation button 62 is held down by the user until the generator 18 indicates a complete seal cycle has been achieved (e.g., via an audible tone, although other indicators, such as a visual indicator or tactile indicator, may be utilized). In certain aspects, at the end of a seal cycle, the generator 18 provides either a first indicator signifying a good seal cycle or a second indicator signifying an incomplete or bad seal cycle.

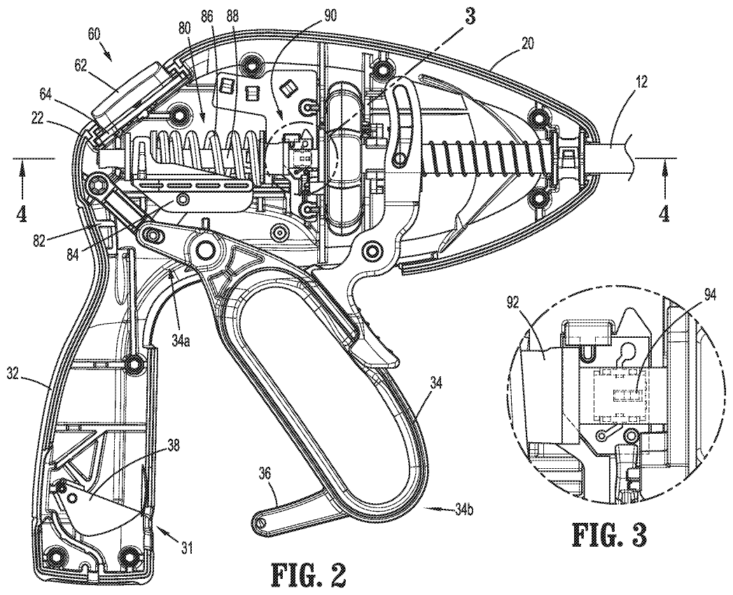

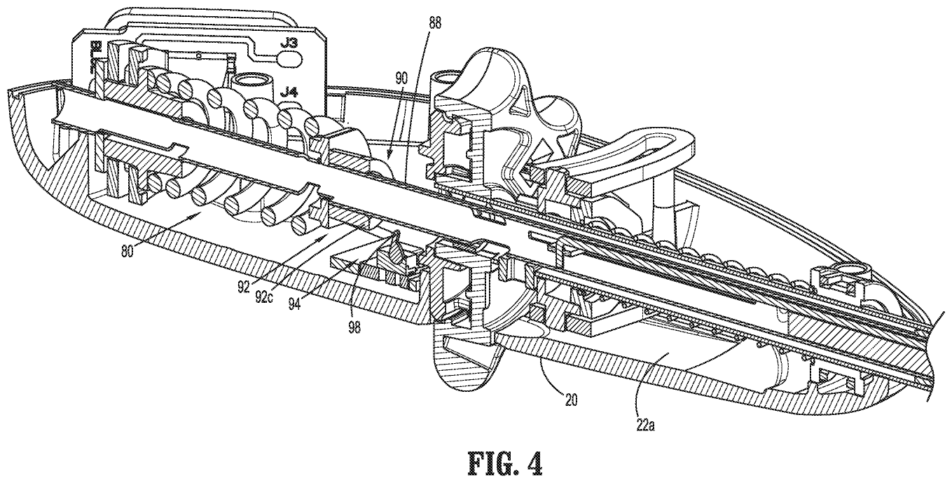

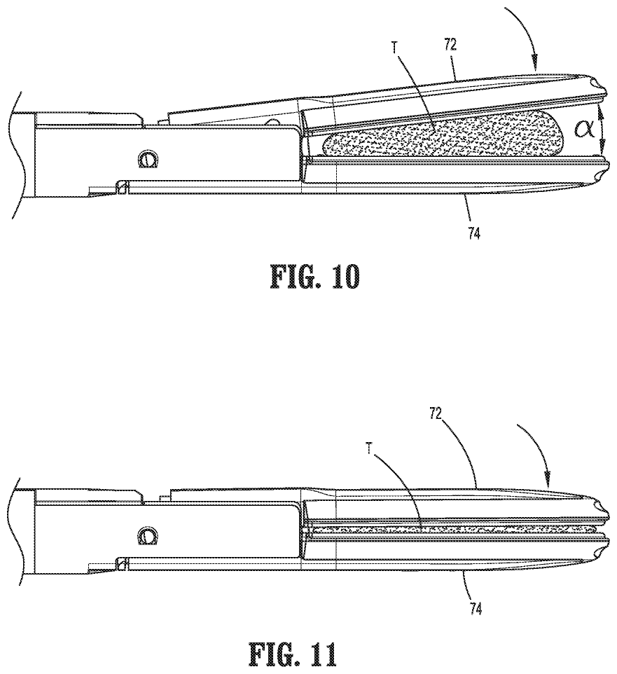

FIGS. 3 and 4 illustrate a tissue sensor assembly 90 of the forceps 10. The tissue sensor assembly 90 is utilized to determine closure of the first and second jaws 72, 74 (FIG. 1) relative to one another to ensure adequate tissue compression between the first and second jaws 72, 74 for tissue (e.g., vessel) sealing. The tissue sensor assembly 90 includes a tissue flag 92 and a tissue flag sensor 94. The tissue flag 92 is coupled to the drive assembly 80 and the tissue flag sensor 94 is coupled to the housing 20. The tissue flag 92 is configured to trigger the tissue flag sensor 94 when the drive tube 88 is moved a predetermined distance corresponding to the first and second jaws 72, 74 (FIG. 10) being closed at or beyond a pre-determined or pre-defined jaw aperture angle “α” (FIG. 10). Specifically, the tissue flag 92 is configured to change the state of the tissue flag sensor 94 at the pre-determined jaw aperture angle “α.” Accordingly, an open position of the first and second jaws 72, 74 corresponds to the tissue flag sensor 94 being in an open state (e.g., the first and second jaws 72, 74 being at an angle greater than the pre-determined jaw aperture angle “α”) and a closed position of the first and second jaws 72, 74 corresponds to the tissue flag sensor 94 being in a closed state (e.g., the first and second jaws 72, 74 being at an angle equal to or less than the pre-determined jaw aperture angle “α”).

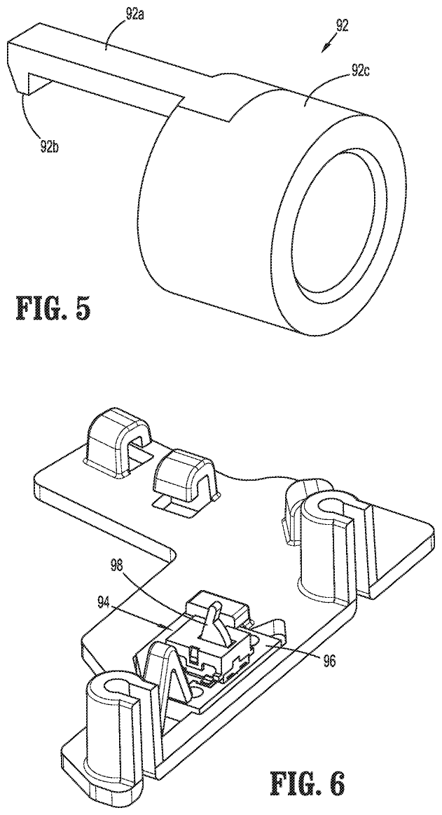

As seen in FIGS. 4 and 5, the tissue flag 92 includes an arm 92a having a finger or flange 92b disposed at a proximal end of the arm 92a that is engaged with the drive assembly 80 such that movement of the drive assembly 80 results in corresponding movement of the tissue flag 92. The tissue flag 92 includes a collar 92c disposed at a distal end of the arm 92a and extending around the drive tube 88. The collar 92c is configured to ride along the drive tube 88 and travel axially with actuation of the drive assembly 80. Specifically, the tissue flag 92 is configured to move distally during closing of the first and second jaws 72, 74 (FIG. 1, e.g., during actuation or squeezing of the movable handle 34) and proximally during opening of the first and second jaws 72, 74 (FIG. 1, e.g., during release of the movable handle 34). The collar 92c is aligned with the tissue flag sensor 94 such that when the pre-determined jaw aperture angle “α” (FIG. 10) is reached, the collar 92c activates the tissue flag sensor 94 and changes the state of the tissue flag sensor 94 from the open state to the closed state. It should be understood that the pre-determined jaw aperture angle “α” can be any angle based on the desired or necessary closure force and/or jaw aperture angle (which dictates the closure force capable of being applied) required to seal tissue. Thus, the length of the tissue flag 92 can be adjusted to change the pre-determined jaw aperture angle “α” at which the tissue flag sensor 94 changes states.

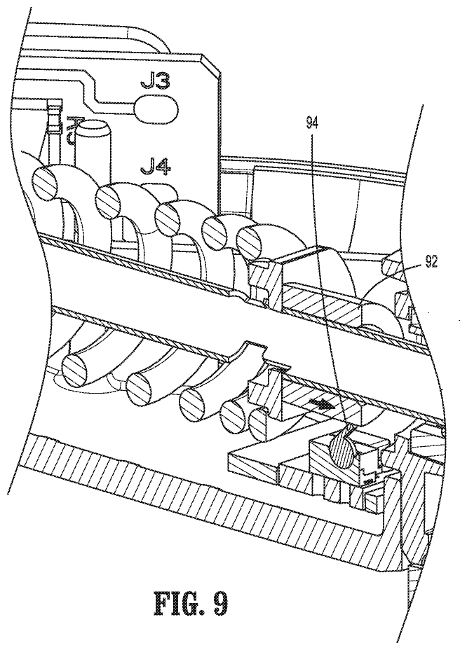

As seen in FIGS. 4 and 6, the tissue flag sensor 94 is secured to an inner wall 22a of the body 22 of the housing 20 in a fixed location within the housing 20. The tissue flag sensor 94 includes a printed circuit board assembly 96 and a switch 98 mounted to and electrically connected to the printed circuit board assembly 96. The printed circuit board assembly 96 is electrically coupled to the main circuit board assembly 64 (FIG. 2), for example, by a wire (not shown). In aspects, the switch 98 is a micro-electromechanical switch configured as a momentary push-to-close toggle that is biased in an open position (FIG. 4) and movable to a closed position (FIG. 9). It should be understood that the tissue flag sensor 94 may be any bi-state sensor configured to detect a single condition and to output a signal corresponding to whether or not that condition exists. It is envisioned, however, that the tissue flag sensor 94 may be a sensor capable of detecting three or more states or of monitoring changing conditions.

As discussed above, the tissue sensor assembly 90 is configured to detect if the first and second jaws 72, 74 have reached the pre-determined jaw aperture angle “α.” As seen in FIGS. 4 and 7, when the first and second jaws 72, 74 are in an open configuration, the drive tube 88 is in a proximal-most position and the tissue flag 92 is disposed proximal to, and in spaced relation from, the tissue flag sensor 94. In this configuration, the switch 98 of the tissue flag sensor 94 is biased in the open position and the tissue flag sensor 94 is in the open state. The tissue sensor assembly 90 maintains the open state of the tissue flag sensor 94 during closure of the first and second jaws 72, 74 until the collar 92c of the tissue flag 92 contacts the switch 98 of the tissue flag sensor 94, as shown in FIG. 8. Upon further distal movement, as shown in FIG. 9, the tissue flag 92 moves the tissue flag sensor 94 to the closed position as the pre-determined jaw aperture angle “α” is reached, as shown in FIG. 10. The tissue flag sensor 94 will register the closed state upon reaching the predetermined jaw aperture angle “α” (e.g., a partially closed configuration of the first and second jaws 72, 74), and will continue to register the closed state through the closed configuration of the first and second jaws 72, 74, as seen in FIG. 11, thereby allowing for grasping and sealing of tissue of different thicknesses.

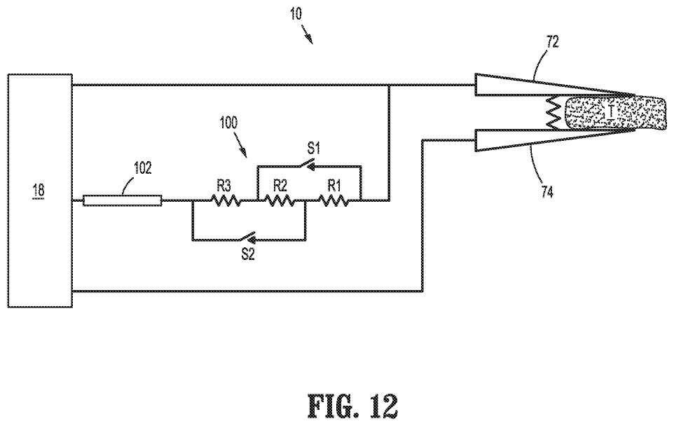

Turning now to FIG. 12, a schematic representation of the forceps 10 and the generator 18 is shown indicating the first and second jaws 72, 74 in an open position and thus, the tissue sensor assembly 90 is in the open state, and the activation assembly 60 (FIG. 2) in an open state (e.g., the activation button 62 is not actuated and no energy is being delivered). The forceps 10 includes an identifier circuit 100. The identifier circuit 100 includes three resistors, a first resistor “R1”, a second resistor “R2”, and a third resistor “R3,” each having a different value that is selected to work with the generator algorithm. The identifier circuit 100 further includes a first switch “S1” and a second switch “S2” for parallel and/or series connection of the first, second, and third resistors “R1”, “R2”, “R3.” The first switch “S1” indicates the state of the tissue sensor assembly 90 (FIG. 2), and the second switch “S2” indicates the state of the activation assembly 60 (FIG. 2). The identifier circuit 100 has an output terminal 102 for communicating the impedance of the identifier circuit 100, referred to herein as the identifier circuit impedance, to the generator 18. In aspects, with momentary additional reference to FIG. 1, the plug 16 of the electrosurgical cable 14 of the forceps 10 includes a printed circuit board assembly (not explicitly shown) including the identifier circuit 100, and the output terminal 102 of the identifier circuit 100 is defined as a pin 17 of the plug 16 of the electrosurgical cable 14 that is connectable to the generator 18.

The identifier circuit 100 enables four states of the output terminal 102 (e.g., the pin 17 (FIG. 1)) of the forceps 10 to be monitored by the generator 18 per the identifier circuit impedance at the output terminal 102. Based on the identifier circuit impedance, the generator 18 determines when sealing energy is being requested by a user, as well as what is happening with respect to the tissue “T” between the first and second jaws 72, 74. Accordingly, the activation assembly 60 provides feedback to the generator 18 regarding the application of energy to the tissue “T” disposed between the first and second jaws 72, 74 (e.g., whether the activation button 62 is activated to supply energy or not), and the tissue sensor assembly 90 provides feedback to the generator 18 regarding the predetermined jaw aperture angle “α” and thus, the thickness of the tissue “T,” through a single output terminal.

The four states “X1”-“X4” of the forceps 10 are identified by the generator 18 based on four identifier circuit impedance values “Z1”-“Z4” (or four ranges of impedance values) read out from the output terminal 102 (e.g., the pin 17 (FIG. 1)) that are switched by the first and second switches “S1,” “S2.” These four states are summarized in Table 1 below:

TABLE 1
Four states of the forceps read through the identifier circuit

| | Activation | Tissue Sensor | Identifier |
|---|---|---|---|
| Forceps | Assembly | Assembly | Circuit |
| State | State (S2) | State (S1) | Impedance |
| X1 | OPEN | OPEN | Z1 |
| X2 | CLOSED | OPEN | Z2 |
| X3 | OPEN | CLOSED | Z3 |
| X4 | CLOSED | CLOSED | Z4 |

When the forceps 10 is in the first state “X1,” both the activation assembly 60 and the tissue sensor assembly 90 are in the open states and thus, the first and second switches “S1,” “S2” are open. In this first state “X1,” the first, second, and third resistors “R1,” “R2,” “R3” are in series and the identifier circuit impedance “Z1” is high. When the forceps 10 is in the second state “X2,” the activation assembly 60 and thus, the second switch “S2,” are in the closed state and the tissue sensor assembly 90 and thus, the first switch “S1,” are in the open state. In this second state “X2,” the identifier circuit impedance “Z2” is that of the first resistor “R1.” When the forceps 10 is in the third state “X3,” the activation assembly 60 and thus, the second switch “S2,” are in open state and the tissue sensor assembly 90 and thus, the first switch “S1,” are in the closed state. In this third state “X3,” the identifier circuit impedance “Z3” is that of the third resistor “R3.” When the forceps 10 is in the fourth state “X4,” both the activation assembly 60 and the tissue sensor assembly 90 are in the closed states and thus, the first and second switches “S1,” “S2” are closed. In this fourth state “X4,” the resistors are in parallel and the identifier circuit impedance “Z4” is low. In aspects, the identifier circuit impedance “Z1” of the forceps 10 in the first state “X1” is the highest impedance value generated by the identifier circuit 100 and the identifier circuit impedance “Z4” in the fourth state “X4” is the lowest impedance valve.

In aspects, after grasping tissue “T” between the first and second jaws 72, 74, energy is delivered to the first and second jaws 72, 74 upon activation of the activation assembly 60 to seal the tissue “T.” During a seal cycle, the generator algorithm monitors and adjusts energy deliver to the tissue “T” based upon reading the tissue impedance, and also monitors the identifier circuit impedance of the identifier circuit 100 to determine if energy was requested/ceased by the user and whether the tissue sensor assembly 90 is in the open or closed state. At the end of the seal cycle, prior to issuing a notification signifying a good seal cycle or a bad seal cycle to a user, the generator algorithm assesses the quality of the seal as well as the status of the tissue sensor assembly 90 via the identifier circuit 100. For a detailed description of an exemplary generator algorithm suitable for determining seal quality, reference may be made to U.S. Pat. No. 8,920,421, the entire contents of which are incorporated by reference herein.

A good seal indication is issued if the generator algorithm detects a complete seal and the tissue sensor assembly 90 is in the closed state. A bad seal indication is issued if the generator algorithm detects a complete seal, but the tissue sensor assembly 90 is in the open state. Accordingly, despite the generator algorithm detecting a complete seal, the status of the tissue sensor assembly 90, if in the open state, inhibits determination of a good seal. This is because at large jaw aperture angles, the likelihood of achieving a good seal is reduced. If the first and second jaws 72, 74 have not achieved the predetermined jaw aperture angle “α” at the end of the seal cycle, it cannot be determined if the first and second jaws 72, 74 were in direct contact with the tissue “T” and/or provided proper seal pressure throughout the entirety of the seal cycle. As such, the ability to confirm a good seal is reduced. In aspects, rather than a bad seal indication in response to the generator algorithm detecting a complete seal with the tissue sensor assembly 90 in the open state, an intermediate indication (differentiated from each of the good and bad seal indications) may be provide such as, for example, a check seal indication or other indication notifying the user that a good seal may not have been achieved. The bad seal indication may also be issued if the generator algorithm detects an incomplete seal (in which case the state of the tissue sensor assembly 90 will not change the outcome). The indication(s) for a good seal, a bad seal, or a potentially bad seal may include audible tones output from the generator, vibrations of the handle of the forceps, LED illumination on the generator and/or forceps, text and/or graphic displays on a GUI of the generator, etc.

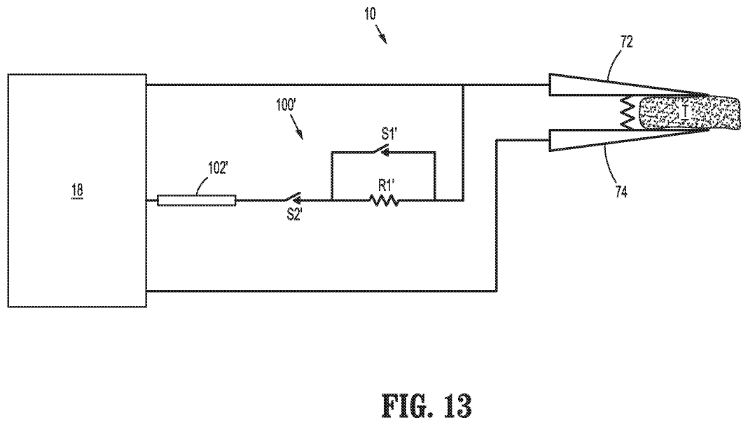

As shown in FIG. 13, an identifier circuit 100′ in accordance with another aspect of this disclosure may be utilized to enable three states of the output terminal 102′ of the forceps 10 to be monitored by the generator 18. In the first state, the second switch “S2′” is open, indicating that the activation assembly 60 (FIG. 1) has not been activated and is in an open state. Accordingly, in the first state, the impedance circuit 100′ is open and the generator 18 can determine that sealing energy is not being requested by a user. In the second and third states, the second switch “S2′” is closed, indicating that the activation assembly 60 has been activated. In the second state, the first switch “S1′” is open, indicating that the tissue sensor assembly 90 is open and the pre-determined jaw aperture angle “α” was not reached, and in the third state, the first switch “S1′” is closed, indicating that the tissue sensor assembly 90 is closed and the pre-determined jaw aperture angle “α” was achieved. Accordingly, the status of the first switch “S1′,” in conjunction with activation of the second switch “S2′,” corresponding with the second and third states of the identifier circuit 100′, outputs a unique voltage signal due to whether the first resistor “R1” is being included in the circuit of the identifier circuit 100′.

In other aspects, the identified circuit 100, 100′ may be utilized to prevent the activation of the activation assembly 60 until the tissue sensor assembly 90 is in the closed state. In yet other aspects of use, the identifier circuit 100, 100′ may be utilized to adjust the delivery of energy to tissue “T” during a seal cycle depending upon the state of the tissue sensor assembly 90.

While illustrated and described as being used as a hand-held electrosurgical device hereinabove, it is contemplated and within the scope of this disclosure for the forceps to be configured for use with other surgical apparatus, such as robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to an operating theater or located in a remote location. In this instance, one team of surgeons or nurses may prep a patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the aspects described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

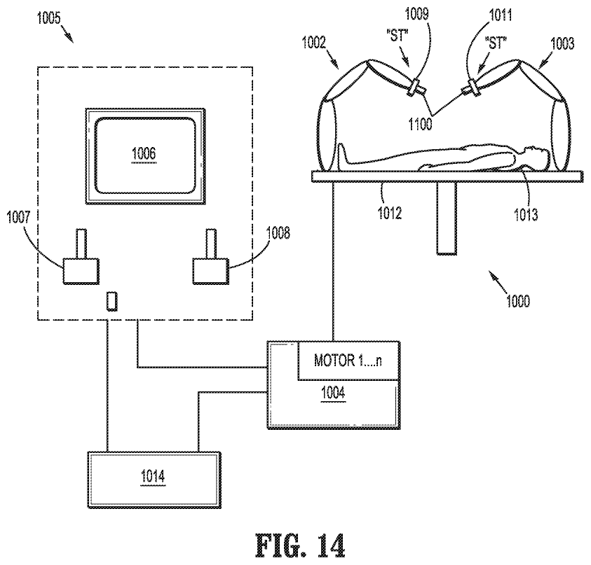

Referring now to FIG. 14, a medical workstation is shown generally as workstation 1000 and generally may include a plurality of robot arms 1002, 1003; a control device 1004; and an operating console 1005 coupled with the control device 1004. The operating console 1005 may include a display device 1006, which may be set up in particular to display three-dimensional images; and manual input devices 1007, 1008, by means of which a person (not shown), for example a surgeon, may be able to telemanipulate the robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100, in accordance with any one of several embodiments disclosed herein, as will be described in greater detail below.

The robot arms 1002, 1003 may be driven by electric drives (not shown) that are connected to the control device 1004. The control device 1004 (e.g., a computer) may be set up to activate the drives, in particular by means of a computer program, in such a way that the robot arms 1002, 1003, their attaching devices 1009, 1011 and thus the surgical tool “ST” (including end effector 1100) execute a desired movement according to a movement defined by means of the manual input devices 1007, 1008. The control device 1004 may also be set up in such a way that it regulates the movement of the robot arms 1002, 1003 and/or of the drives.

The medical workstation 1000 may be configured for use on a patient 1013 lying on a patient table 1012 to be treated in a minimally invasive manner by means of the end effector 1100. The medical workstation 1000 may also include more than two robot arms 1002, 1003, the additional robot arms likewise being connected to the control device 1004 and being telemanipulatable by means of the operating console 1005. A medical instrument or surgical tool (including an end effector 1100) may also be attached to the additional robot arm. Medical workstation 1000 may include a database 1014, in particular coupled to the control device 1004, in which are stored, for example, pre-operative data from the patient/living being 1013 and/or anatomical atlases.

Aspects of this disclosure may be further described by reference to the following numbered paragraphs:
1. A surgical instrument comprising:
   a housing;
   a shaft extending distally from the housing;
   an end effector assembly disposed at a distal end of the shaft, the end effector assembly including a first jaw and a second jaw movable relative to one another between an open configuration wherein the first and second jaws define a first jaw aperture angle greater than a pre-determined jaw aperture angle and a closed configuration wherein the first and second jaws define a second jaw aperture angle less than the pre-determined jaw aperture angle;
   a drive assembly including a drive tube extending through the shaft and configured to move the first and second jaws between the open and closed configurations; and
   a tissue sensor assembly including:
      a tissue flag coupled to the drive assembly and translatable with the drive tube; and
      a tissue flag sensor coupled to the housing for detecting movement of the tissue flag.
2. The surgical instrument according to paragraph 1, wherein the tissue sensor assembly is configured to change states when the first and second jaws are moved between the open and closed configurations.
3. The surgical instrument according to paragraph 1, wherein the tissue sensor assembly is in an open state when the first and second jaws are in the open configuration, and the tissue sensor assembly is in a closed state when the first and second jaws are in the closed configuration.
4. The surgical instrument according to paragraph 1, wherein, when the first and second jaws are in the open configuration, the tissue flag is disposed in spaced relation from the tissue flag sensor and, which the first and second jaws are in the closed configuration, the tissue flag contacts the tissue flag sensor.
5. The surgical instrument according to paragraph 1, further comprising an identifier circuit configured to identify when the first and second jaws are in the open configuration or the closed configuration.
6. The surgical instrument according to paragraph 5, further comprising an activation assembly configured to deploy energy to the first and second jaws.
7. The surgical instrument according to paragraph 6, wherein the identifier circuit is configured to identify when energy is deployed to the first and second jaws.
8. The surgical instrument according to paragraph 7, wherein an output of the identifier circuit is used to determine the quality of a seal generated by the activation assembly.
9. The surgical instrument according to paragraph 7, wherein the identifier circuit enables four states of the surgical instrument to be monitored based on an open state or a closed state of each of the tissue sensor assembly and the activation assembly.
10. The surgical instrument according to paragraph 9, wherein the identifier circuit includes a plurality of resistors, a first switch associated with the tissue sensor assembly, and a second switch associated with the activation assembly, wherein the first and second switches determine a connection scheme of the plurality of resistors.
11. The surgical instrument according to paragraph 10, wherein the identifier circuit generates an identifier circuit impedance based on each of the four states of the surgical instrument.
12. The surgical instrument according to paragraph 11, wherein the identifier circuit impedance is read through an output terminal of the identifier circuit.
13. The surgical instrument according to paragraph 12, wherein the output terminal is configured to communicate with a generator.
14. The surgical instrument according to paragraph 12, further comprising an electrosurgical cable extending from the housing, the electrosurgical cable including a plug connectable to a generator and the output terminal is a pin of the plug.
15. A surgical instrument, comprising:
   a housing;
   a shaft extending distally from the housing;
   an end effector assembly disposed at a distal end of the shaft, the end effector assembly including a first jaw and a second jaw movable relative to one another between an open configuration wherein the first and second jaws define a first jaw aperture angle greater than a pre-determined jaw aperture angle and a closed configuration wherein the first and second jaws define a second jaw aperture angle less than the pre-determined jaw aperture angle;
   a tissue sensor assembly having an open state when the first and second jaws are in the open configuration and a closed state when the first and second jaws are in the closed configuration;
   an activation assembly having an open state when no energy is deployed to the first and second jaws and a closed state when energy is deployed to the first and second jaws; and
   an identifier circuit configured to identify four states of the surgical instrument based on the open state or the closed state of each of the tissue sensor assembly and the activation assembly.
16. The surgical instrument according to paragraph 15, wherein the identifier circuit includes a plurality of resistors, a first switch associated with the tissue sensor assembly, and a second switch associated with the activation assembly, wherein the first and second switches determines a connection scheme of the plurality of resistors.
17. The surgical instrument according to paragraph 15, wherein the identifier circuit outputs an identifier circuit impedance based on each of the four states of the surgical instrument.
18. The surgical instrument according to paragraph 17, wherein the identifier circuit impedance is used to determine the quality of a seal generated by the activation assembly.
19. The surgical instrument according to paragraph 15, further including a drive assembly configured to move the first and second jaws between the open and closed configurations, and the tissue sensor assembly includes a tissue flag movably coupled to the drive assembly and a tissue flag sensor for detecting movement of the tissue flag.
20. The surgical instrument according to paragraph 19, wherein the tissue flag is aligned with the tissue flag sensor such that when the first and second jaws are in the open configuration, the tissue flag is disposed in spaced relation from the tissue flag sensor and, which the first and second jaws are in the closed configuration, the tissue flag contacts the tissue flag sensor.

While aspects of the disclosure have been shown in the drawings, it is not intended that the disclosure be limited thereto, as it is intended that the disclosure be as broad in scope as the art will allow and that the specification be read likewise. It is to be understood, therefore, that the disclosure is not limited to the precise aspects described, and that various other changes and modifications may be made by one skilled in the art without departing from the scope or spirit of the disclosure. Additionally, the elements and features shown and described in connection with certain aspects of the disclosure may be combined with the elements and features of certain other aspects without departing from the scope of the disclosure, and that such modifications and variation are also included within the scope of the disclosure. Therefore, the above description should not be construed as limiting, but merely as exemplifications of aspects of the disclosure. Thus, the scope of the disclosure should be determined by the appended claims and their legal equivalents, rather than by the examples given.

1. A surgical instrument comprising:
a housing;
a shaft extending distally from the housing;
an end effector assembly disposed at a distal end of the shaft, the end effector including a first jaw and a second jaw movable relative to one another between an open configuration wherein the first and second jaws define a first jaw aperture angle greater than a predetermined jaw aperture angle and a closed configuration wherein the first and second jaws define a second jaw aperture angle less than the pre determined jaw aperture angle;
a drive assembly including a drive tube extending through the shaft and configured to move the first and second jaws between the open and closed configurations; and a tissue sensor assembly including:
a tissue flag coupled to the drive assembly and translatable with the drive tube; and a tissue flag sensor coupled to the housing for detecting movement of the tissue flag.

2. The surgical instrument according to claim 1, wherein the tissue sensor assembly is configured to change states when the first and second jaws are moved between the open and closed configurations.

3. The surgical instrument according to claim 1, wherein the tissue sensor assembly is in an open state when the first and second jaws are in the open configuration, and the tissue sensor assembly is in a closed state when the first and second jaws are in the closed configuration.

4. The surgical instrument according to claim 1, wherein, when the first and second jaws are in the open configuration, the tissue flag is disposed in spaced relation from the tissue flag sensor and, when the first and second jaws are in the closed configuration, the tissue flag contacts the tissue flag sensor.

5. The surgical instrument according to claim 1, further comprising an identifier circuit configured to identify when the first and second jaws are in the open configuration or the closed configuration.

6. The surgical instrument according to claim 5, further comprising an activation assembly configured to deploy energy to the first and second jaws.

7. The surgical instrument according to claim 6, wherein the identifier circuit is configured to identify when energy is deployed to the first and second jaws.

8. The surgical instrument according to claim 6, wherein an output of the identifier circuit is used to determine the quality of a seal generated by the activation assembly.

9. The surgical instrument according to claim 6, wherein the identifier circuit enables four states of the surgical instrument to be monitored based on an open state or a closed state of each of the tissue sensor assembly and the activation assembly.

10. The surgical instrument according to claim 9, wherein the identifier circuit includes a plurality of resistors, a first switch associated with the tissue sensor assembly, and a second switch associated with the activation assembly, wherein the first and second switches determine a connection scheme of the plurality of resistors.

11. The surgical instrument according to claim 10, wherein the identifier circuit generates an identifier circuit impedance based on each of the four states of the surgical instrument.

12. The surgical instrument according to claim 11, wherein the identifier circuit impedance is read through an output terminal of the identifier circuit.

13. The surgical instrument according to claim 12, wherein the output terminal is configured to communicate with a generator.

14. The surgical instrument according to claim 12, further comprising an electrosurgical cable extending from the housing, the electrosurgical cable including a plug connectable to a generator and the output terminal is a pin of the plug.

15. A surgical instrument, comprising:
a housing;
a shaft extending distally from the housing;
an end effector assembly disposed at a distal end of the shaft, the end effector including a first jaw and a second jaw movable relative to one another between an open
configuration wherein the first and second jaws define a first jaw aperture angle greater than a predetermined jaw aperture angle and a closed configuration wherein the first and second jaws define a second jaw aperture angle less than the pre determined jaw aperture angle;
a tissue sensor assembly having an open state when the first and second jaws are in the open configuration and a closed state when the first and second jaws are in the closed configuration;
an activation assembly having an open state when no energy is deployed to the first and second jaws and a closed state when energy is deployed to the first and second jaws; and
an identifier circuit configured to identify four states of the surgical instrument based on the open state or the closed state of each of the tissue sensor assembly and the activation assembly.
